(19) United States
(12) Patent Application Publication
(10) Pub. No.: US 2007/0142742 A1
(43) Pub. Date: Jun. 21, 2007

(54) METHODS AND SYSTEMS FOR SEGMENTAL LUNG DIAGNOSTICS

(75) Inventors: NIKOLAI ALJURI, Revere, MA (US); Jose G. Venegas, Swapscott, MA (US); Rodney C. Perkins, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PULMONx, Palo Alto, CA (US)

(21) Appl. No.: 11/550,660

(22) Filed: Oct. 18, 2006

Related U.S. Application Data
(63) Continuation of application No. PCT/US06/27478, filed on Jul. 13, 2006.
(63) Continuation-in-part of application No. 11/296,951, filed on Dec. 7, 2005.
(60) Provisional application No. 60/699,289, filed on Jul. 13, 2005.

(51) Int. Cl. A61B 5/08

(52) U.S. Cl. 600/538; 600/529

(57) ABSTRACT

Minimally invasive systems and methods are provided for diagnosing conditions in target lung compartments. Using catheters capable of isolating the target lung compartments and measuring one or more of collateral ventilation, pressure, flow rate, and volume, conditions such as hyperinflation, compliance, gas exchange including oxygen uptake, directionality of collateral channels, blood flow, and blood flow per unit lung volume may be assessed.

### CROSS-REFERENCES TO RELATED APPLICATIONS

This application is a continuation of PCT/US 06/27478 (Attorney Docket No. 017534-003010PC) filed Jul. 13, 2006, which claimed the benefit of U.S. Provisional No. 60/699,289 (Attorney Docket No. 017534-003000US), filed on Jul. 13, 2005, and is a continuation-in-part of U.S. application Ser. No. 11/296,951 (Attorney Docket No. 017534-002820US), filed on Dec. 7, 2005, the full disclosures of which are incorporated herein by reference.

### BACKGROUND OF THE INVENTION

1. Field of the Invention

The present invention relates generally to respiratory medicine and more specifically to the field of assessing lung condition and function in isolated lung compartments.

The lungs comprise a plurality of bronchopulmonary compartments, referred to hereinafter as “lung compartments,” which are separated from one another by a double layer of infolded reflections of visceral pleura called the “fissures.” The fissures which separate the lung compartments are typically impermeable and the lung compartments receive and expel air only through the upper airways which open into the compartments. While the compartments within particular lung lobules can communicate with each other through well-known collateral pathways, such as the inter-bronchiolar Martin's Channels, the bronchiole-alveolar channels of Lambert, and the inter-alveolar pores of Kohn, such pathways are generally not thought to pass through the impermeable fissures that separate the lung compartments. Recent studies have shown, however, that the interlobar fissures are not always complete, and therefore the lobular regions of the lungs may be connected and provide a pathway for collateral airflow or inter-lobular collateral ventilation. Significantly, the presence of such collateral pathways between lung compartments is markedly increased in emphysema patients.

Because of recent advances in the treatment of chronic obstructive pulmonary disease (COPD) there has been a heightened interest in collateral ventilation. Various COPD treatments involve the removal of trapped air to reduce the debilitating hyperinflation caused by the disease and occlusion of the feeding bronchus to maintain the area at a reduced volume. The concept guiding these approaches is that aspiration and/or absorption atelectasis of emphysematous lung regions can reduce lung volume without the need to remove tissue. One such type of COPD treatment is Endobronchial Volume Reduction (EVR) uses a catheter-based system to reduce lung volume. With the aid of fiberoptic visualization and specialty catheters, a physician can selectively collapse a segment or segments of the diseased lung. An occlusal stent is then positioned within the lung segment to prevent the segment from re-inflating.

FIGS. 1A-1C illustrate an example of such an EVR procedure targeting the right upper lobe RUL of the right lung RL of a patient. Here, the right upper lobe RUL is hyperinflated. A catheter 2 is advanced through the trachea T into the lung passageways feeding the right upper lobe RUL. The right upper lobe RUL is then reduced in volume, as illustrated in FIG. 1B, and a plug, valve or occlusal stent 4 is placed within the lung passageway reducing the volume of the right upper lobe RUL. However, as shown in FIG. 1C, collateral channels CH may be present connecting the right upper lobe RUL with the right middle lobe RML and/or the right lower lobe RLL. Consequently, the EVR may only be temporarily successful as the right upper lobe RUL re-expands or re-hyperinflates due to refill through the collateral channels CH over time. In some instances, a desired volume reduction may be impossible due to air being drawn in from neighboring lobes via the collateral channels CH.

FIGS. 2A-2B schematically illustrate collateral channels CH in the right lung RL. FIG. 2A illustrates a variety of inter-lobar collateral channels CH between the right upper lobe RUL, right middle lobe RML and right lower lobe RLL. FIG. 2B illustrates intra-lobar or inter-segmental collateral channels CH which connect individual lung segments (e.g. S, S₁, S₂) within the lung lobes. These inter-segmental collateral channels allow the periphery of each of the lung compartments to communicate with one another and include well-known collateral pathways such as Martin's Channels, pores of Kohn and Lambert's canals.

A method of measuring inter-compartment collateral ventilation has been to measure resistance to collateral ventilation (R_{coll}). Assessment of the relationship between steady-state flow through collateral channels (Q_{coll}) and the pressure drop across them is a direct way for measuring the resistance to collateral ventilation (R_{coll}). Many investigators have attempted to use this approach in the past but the most simple and versatile way to make this measurement was first described by Hilpert (Hilpert P. Kollaterale Ventilation Habilitationsschirift, aus der Medizinischen. Tubingen, West Germany: Tubingen Universitatsklinik, 1970. Thesis). This method is schematically illustrated in FIGS. 3A-3C and includes injecting a constant flow of air Q_{coll} as illustrated in FIG. 3B to a target area or sealed target compartment Cₛ. Q_{coll} is supplied by a flow generator 5 through a double-lumen isolation catheter 6 having an isolation cuff 7 which is wedged into a peripheral airway and seals the compartment Cₛ. Q_{coll} is injected through one lumen of the isolation catheter 6 while air pressure P_{b} at the site of airway obstruction is measured through the other lumen as illustrated in FIG. 3C. Under steady-state conditions, the ratio of P_{b} over Q_{coll} provides a quantitative measure for the resistance to collateral ventilation, which includes the resistance in the collateral channels R_{coll} and the resistance in the small airways R_{saw} of the isolated compartment between the collateral channels and the distal end of the catheter 6. This technique can be somewhat useful as an experimental tool, however it has significant limitations experimentally and its clinical use poses an additional risk to the patient. Namely, applying a constant air flow to a diseased area of the lung can be hazardous if not done correctly. For example in the presence of bullous emphysema, the pressure could enlarge the bullae or create new bulla, or could lead to increased hyperinflation or pneumothorax.

Another method that imposes lesser risk to the patient, relatively to Hilpert's method, has been described by Woolcock and Macklem (Woolcock, A. J, and P. T. Macklem. Mechanical factors influencing collateral ventilation in human, dog, and pig lungs. J. Appl. Physiol. 30:99-115, 1971). This method involves the rapid injection of an air bolus beyond the wedged catheter into the target lung segment, and the rate at which pressure falls as the obstructed segment empties into the surrounding lung through collateral channels is governed by the time constant for collateral ventilation τ_{coll} (the time it takes for the pressure change produced by the air bolus injection to drop to about 37 percent of its initial value). Here R_{coll} is indirectly measured as the ratio between τ_{coll} and the compliance of the target segment Cₛ. Calculations of R_{coll} via this method, however, are highly dependent on several questionable assumptions, including homogeneity within the obstructed segment and in the surrounding lung.

The previously described methods for assessing collateral ventilation would suffer from a number of drawbacks. The Woolcock and Macklem method is generally unsuitable for assessing collateral ventilation while the patient is breathing or under conditions similar to those in which the lung compartment has already been targeted for treatment. The values for collateral resistance obtained by the methods described above generally range from 10⁻¹ to 10⁺2 cmH₂O/(ml/s) for normal human lungs and from approximately 10⁻³ to 10⁻¹ cmH₂O/(ml/s) for emphysematous human lungs.

The presence of inter-compartmental collateral ventilation can also be assessed by isolation of a target lung compartment and subsequent introduction of Heliox (21% O₂/79% He) or other tracer gas. Detection of tracer gas in the target segment indicates the presence of collateral channels allowing gas to flow from the surrounding lung into the target lung segment. The technique does not provide for quantifying the amount of collateral flow or the collateral resistance.

Experimental attempts to detect the presence of inter-compartmental collateral ventilation in excised, deflated lungs rely on cannulating, sealing, and insufflating the lung with air while separate neighboring lung regions are concurrently sealed. Those neighboring regions which inflate are determined to have collateral channels allowing the inflow of the air. Such techniques are not directly applicable to human subjects.

U.S. Patent Application 2003/0228344 Al describes a one-way valve which is placed in an airway feeding a targeted lung compartment. The one-way valve allows air to pass out of the compartment but not into the compartment. If atelectasis (loss of gas from the isolated lung compartment), eventually is observed, the lung compartment is diagnosed as being free from collateral channels (at least those which permit the inflow of gas from adjacent lung compartments into the target lung compartment). If atelectasis is not observed, it is assumed that collateral channels exist which permit the inflow of air to the target compartment from surrounding compartments. While generally identifying lung compartments which are subject to the inflow of gas via collateral channels, the techniques described in this patent application are not able to quantify the amount of collateral ventilation or the value of collateral resistance.

For these reasons, a direct, accurate, simple and minimally invasive methods for assessing collateral ventilation and/or collateral resistance between lung compartments would be desirable. In addition to detecting and measuring collateral ventilation, other techniques for diagnosing lung compartments, including determining hyperinflation, measuring gas exchange, typically oxygen uptake, determining the directionality of collateral channels (into or away from a target lung compartment), and assessing blood flow and/or blood flow per unit lung volume in a target lung compartment, would be desirable. At least some of these objectives will be met by the invention described below.

### BRIEF SUMMARY OF THE INVENTION

Minimally invasive methods, systems and devices are provided for qualitatively and quantitatively assessing the condition and function of individual lung compartments, including the extent of hyperinflation of a lung compartment, compliance of a lung compartment, efficiency of gas exchange within a lung compartment such as the value of oxygen uptake within a lung compartment, the directionality of collateral flow channels between adjacent lung compartments, and the rate or degree of blood flow and/or blood flow/unit of volume within a lung compartment. The methods, systems, and devices generally rely on accessing, isolating, and at least partially occluding a target lung compartment within the lung of a living patient in order to perform the diagnostic protocol. Typically, a lung of the patent is accessed by advancement of a catheter through the tracheobronchial tree to an airway, typically referred to as feeding bronchus, which feeds the target lung compartment. The airway is usually occluded by an expansible occlusion member, typically a balloon on the catheter, and a variety of measurements may be taken with or through the catheter in a manner which presents a minimum risk to the patient.

The methods, systems, and devices of the present invention allow a patient to be diagnosed and for the diagnostic information to be used in selecting treatment options. For example, determinations of hyperinflation, compliance, oxygen uptake, blood flow, and/or blood flow per unit lung volume, generally relate to the health of a particular lung compartment. Lung compartments which appear to be as healthy as or more healthy than other lung compartments within the lung will generally not be targets for treatment, particularly those treatments which rely on occlusion and volume reduction of a target lung compartment, either by aspiration, atelectasis, or combination of both. Determination of collateral ventilation and/or the direction of flow through collateral channels is a direct predictor of the success of lung volume reductions which rely on occlusion. If flow through the collateral channels allow air to collectively enter the target lung compartment when occluded, the success of such treatments is unlikely.

In a first aspect of the present invention, methods are provided for determining the extent of hyperinflation of a lung compartment, typically in the absence of collateral channels. The lung compartment is occluded, typically with a catheter having a balloon or other expandable occlusion element placed at the upper airway feeding the compartment. As the patient continues normal respiration, air is expelled from the compartment and passes out through the catheter, typically through a one-way valve or other structure which prevents air from passing back into the isolated lung compartment. The total amount of air expelled from the compartment from the time of initial occlusion is measured, and the measured amount of total air is directly proportional to the extent of hyperinflation of the lung compartment. Usually, the amount of expelled air will be measured from the time of initial occlusion until the flow of air expelled from the compartment substantially stops, indicating that excess volume in the lung has been collapsed by the external pressure of the surrounding lung compartments as illustrated in FIG. 13. The flow rate of air expelled from the compartment will typically be monitored, for example, using any conventional flow measurement apparatus, so that a determination can be made of when the air flow substantially stops. Typically, the air volume will be assessed simply by integration of the air flow measurement. It will appreciated that this method for directly determining the extent of hyperinflation will usually be less accurate in lung compartments having collateral flow channels which allow airflow into the lung compartment from adjacent lung compartments. Consequently, if collateral flow channels are present, the amount of expelled air can be measured from the time of initial occlusion until the flow of air expelled from the compartment reaches a steady state as illustrated in FIG. 14 where the observed steady-state flow represents the mean collateral airflow into the lung compartment from adjacent lung compartments. As a result, the flow rate of air due to collateral ventilation can be subtracted from the flow rate of air expelled from the compartment to characterize the extent of excess volume which has been collapsed in the occluded lung compartment by the external pressure of the surrounding lung compartments. FIG. 15 exemplifies the dependency of measured excess air volume on varying degrees of collateral ventilation characterized by a plurality of measured collateral resistance (R_{coll}) values. Collateral ventilation is practically non-existent at high values of R_{coll}, i.e. R_{coll}>100, and almost complete at R_{coll} values three orders of magnitude smaller, i.e. R_{coll} <0. 1; however, there is a wide range of 0. 1<R_{coll}<100 where substantial volume reduction can still take place. For instance, at R_{coll}=1 approximately 50% volume reduction can be expected and therefore a total degree of hyperinflation of roughly twice the measured excess volume. Consequently, it will be appreciated that the degree of hyperinflation can still be determined from the measured excess air volume in the presence of collateral channels, though indirectly, if R_{coll} is known. Methods for determining the directionality of collateral channels are described below.

In a second aspect of the present invention, methods are provided for determining the compliance of an isolated lung compartment by measuring a characteristic pressure-volume curve of the isolated lung compartment as illustrated in FIG. 16. Methods for measuring changes in volume of the isolated lung are described above. Changes in elastic recoil pressure will be obtained from the difference between pressure changes within the isolated lung compartment and changes in pleural pressure. The pressure in the isolated compartment will typically be monitored, for example, using any conventional pressure sensor communicating with the catheter's inside lumen during occlusion of entry of air back into the compartment. Pleural pressure will typically be monitored, for example, using any conventional pressure sensor communicating with an esophageal balloon catheter placed in the subject's esophagus. Usually, the pressures and amount of expelled air will be measured from the time of initial occlusion until the flow of air expelled from the compartment substantially stops or reaches a steady state, indicating that excess volume in the lung has been collapsed by the external pressure of the surrounding lung compartments.

In a third aspect of the present invention, methods are provided for determining the rate of oxygen uptake from an isolated lung compartment. A target lung compartment is occluded, typically with a catheter which allows air to be expelled from the compartment but which substantially blocks or occludes the entry of air back into the compartment. After air flow from the target lung compartment through the catheter ceases, the pressure of air remaining within the compartment may be measured over time. A decrease in the air pressure represents a measure or value of oxygen consumption in the lung compartment since it is only through oxygen exchange with the blood that the gas volume or pressure will be reduced.

Typically, occluding the lung compartment will comprise expanding a balloon or other expandable occlusion structure on the catheter at the airway which feeds the lung compartment. The catheter will typically comprise a one-way valve which allows the air to be expelled from the compartment while blocking or inhibiting the air from entering the compartment. Air pressure will typically be measured with a transducer on the catheter. It will be appreciated that these methods for determining oxygen uptake may be less accurate or inapplicable to lung compartments having collateral channels which permit air flow from adjacent lung compartments into the target lung compartment.

In a fourth aspect of the present invention, the directionality of collateral channels communicating between a target lung compartment and an adjacent lung compartment comprise isolating the target lung compartment so that there is no flow in or out through the connecting upper airway. Pressure within the isolated lung compartment is measured over a plurality of respiratory cycles, and an increase in pressure indicates that collateral channels exist and that those channels have a higher resistance to outflow of gas from the target compartment to adjacent compartment(s) than inflow of gas from the adjacent compartment(s) to the target compartment. Such channels will allow a net inflow of air over time. Conversely, a decrease in pressure in the isolated lung compartment over a plurality of respiratory cycles indicates that the collateral channels exist and have a lower resistance to outflow than to inflow. Such channels will allow a net outflow of air from the target compartment over time.

Isolating the target lung compartment typically comprises expanding an occlusion structure, such as a balloon, on a catheter in the airway leading to the target lung compartment. Pressure is typically measured with a transducer on the catheter. Methods for determining the existence and directionality of collateral flow channels are useful for a number of purposes, including determining the applicability of the methods for measuring hyperinflation and for determining oxygen uptake described above. The methods are also useful for determining whether lung volume reduction treatments relying on occlusion and isolation of the target lung compartment will likely be successful. Such occlusion-based protocols are generally suitable for those patients where the target lung compartment either has no collateral flow channels or where the collateral flow channels have a higher resistance to air inflow than air outflow. It would appreciated in those patients having collateral flow channels which have a lower resistance to air inflow, occlusion of the target lung compartment will not prevent the compartment from re-inflating as air enters from adjacent lung compartments.

In a fifth aspect of the present invention, blood flow and/or blood flow per unit lung volume in a target lung compartment may be assessed by first isolating the lung compartment. A marker is injected into systemic circulation, where the marker has low solubility so that it will be rapidly released into the lung. After the marker reaches an equilibrium distribution in the blood, typically taking from 10 to 15 seconds, a first concentration of the marker in the lung compartment is measured and a second concentration of the marker in another part of lung (or the entire lung other than the isolated compartment) are measured. The first and second marker concentrations may then be compared. A lower gas concentration in the target lung compartment than in the remaining portion(s) of the lung indicates that the target lung compartment is less efficient at exchanging gas with the circulating blood, further indicating that the target lung compartment is likely diseased and more likely candidate to receive lung volume reduction or other therapies. Conversely if the gas concentration of the marker in the lung compartment is at least as high as the marker concentration in the remaining portion(s) of the lung, than the target lung compartment is less likely to be more diseased than the remaining portions of the lung, and less likely to benefit from a therapeutic protocol.

The marker is injected preferably during apnea at mean lung volume. A preferred marker comprises sulfur hexafluoride, and the second concentration may be measured in any compartment of the lung, or more often gas exhaled from the rest of the lung. As with previous test protocols, measurement of the blood flow in the lung will be less accurate or in some cases inapplicable when the lung is compromised by air flow into the lung through collateral channels from adjacent lung compartments.

### BRIEF DESCRIPTION OF THE DRAWINGS

FIGS. 1A-1C illustrate an example of an EVR procedure targeting the right upper lobe of the right lung of a patient.

FIGS. 2A-2B schematically illustrate example collateral channels in the right lung.

FIGS. 3A-3C schematically illustrates a method of supplying constant positive pressure of air to a target compartment.

FIGS. 4A-4D illustrate an embodiment of a minimally invasive method in which a catheter is advanced to the feeding bronchus of a target compartment.

FIGS. 5A-5D, 6 illustrate embodiments of a catheter connected with an accumulator.

FIGS. 7A-7B depict a graphical representation of a simplified collateral system of a target lung compartment.

FIGS. 8A-8C illustrate measurements taken from the system of FIGS. 7A-7B.

FIGS. 9A-9C illustrate a circuit model representing the system of FIGS. 7A-7B.

FIGS. 10A-10B illustrate measurements taken from the system of FIGS. 7A-7B.

FIGS. 11A-11D illustrate graphical comparisons yielded from the computational model of the collateral system illustrated in FIGS. 7A-7B and FIGS. 9A-9B.

FIG. 12A illustrates a two-compartment model which is used to generate a method quantifying the degree of collateral ventilation.

FIG. 12B illustrates an electrical circuit analog model.

FIGS. 12C-12E illustrate the resulting time changes in volumes, pressures and gas concentrations in the target compartment and the rest of the lobe.

FIG. 13 is a graph showing the measured flow rate and expelled volume from an isolated compartment over time in the absence of collateral channels.

FIG. 14 is a graph showing the measured flow rate, reduced excess volume, and measured collateral resistance of air from an isolated compartment over time in the presence of collateral flow.

FIG. 15 is a graph showing the relationship between collateral resistance and excess volume reduction in an isolated lung compartment.

FIG. 16 is a graph showing the relationship between changes in elastic recoil pressure and changes in volume in an isolated lobe.

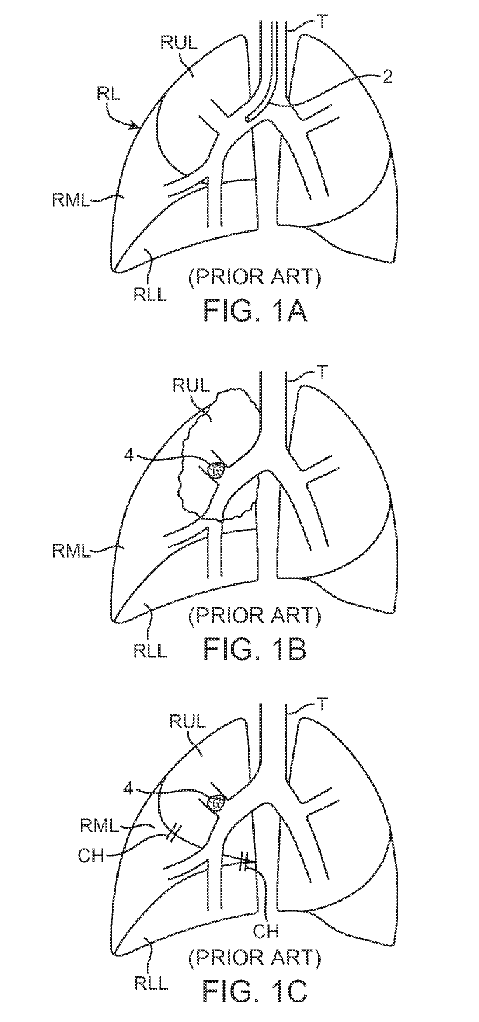

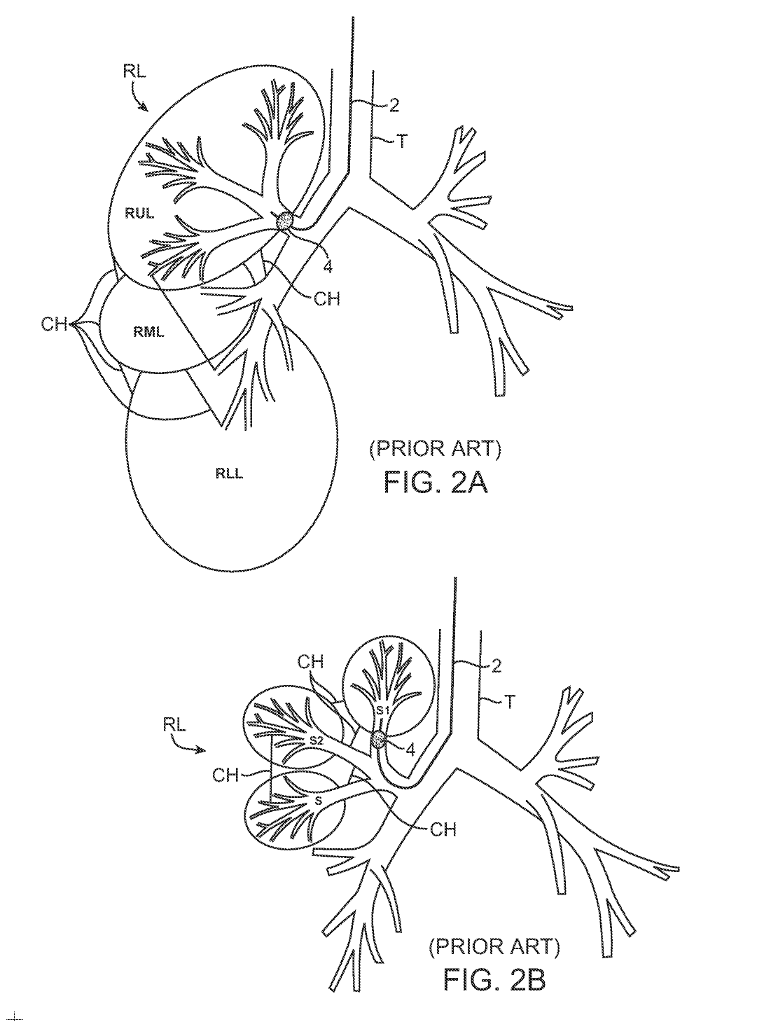

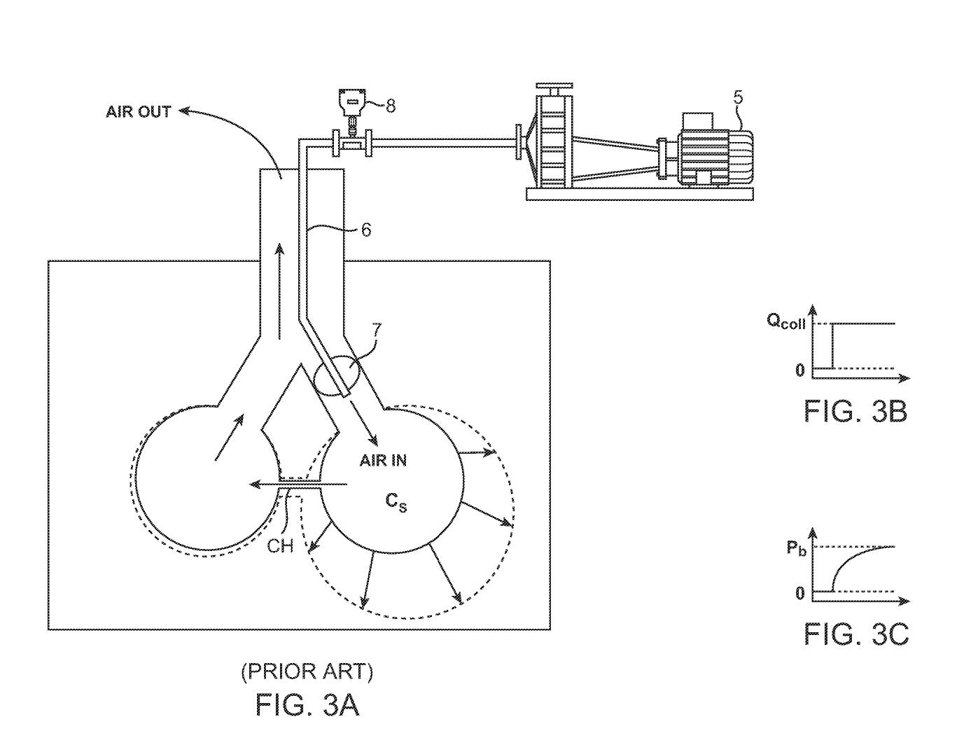

### DETAILED DESCRIPTION OF THE INVENTION

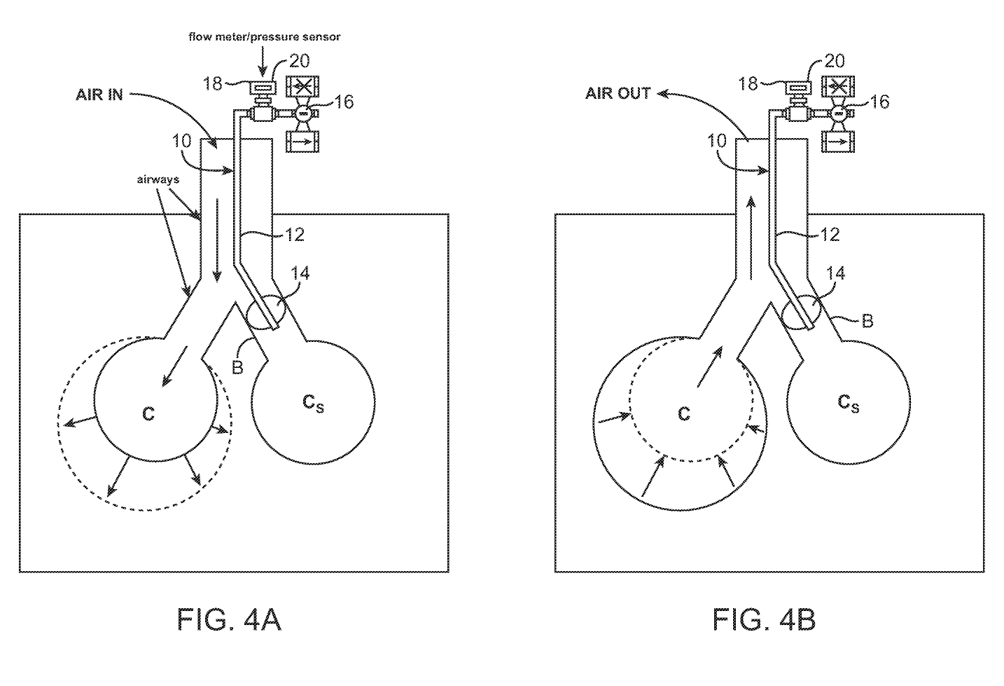

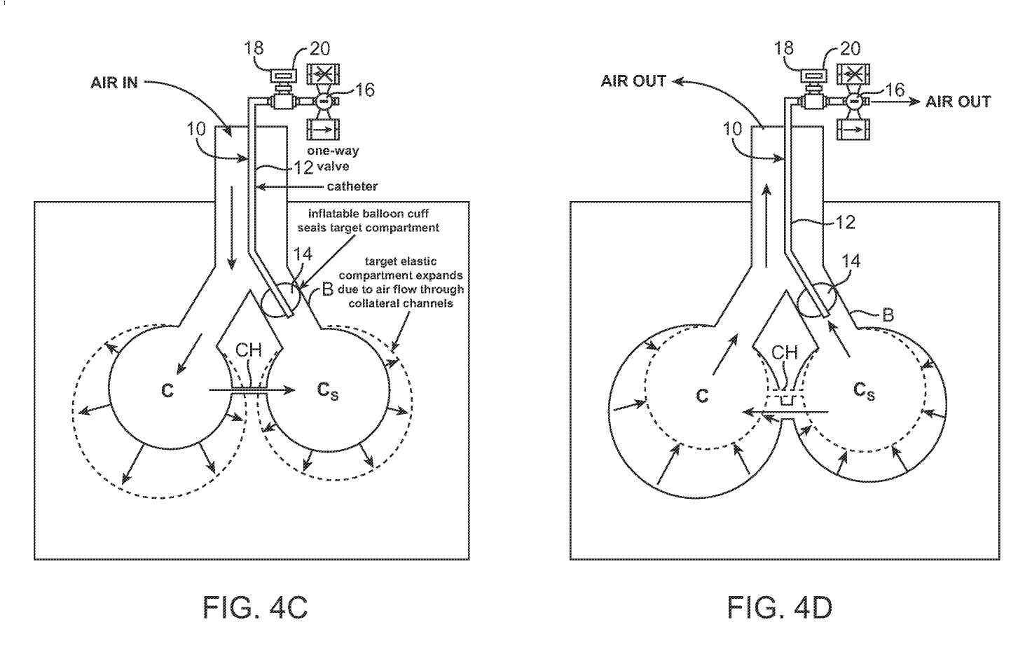

Minimally invasive methods, systems and devices are provided for qualitatively and quantitatively assessing lung condition and function, particularly in target lung compartments or segments which have been isolated from the remainder of the lung. FIGS. 4A-4D illustrate a system which can be utilized for performing the various diagnostic protocols of the present invention and includes a catheter 10 which may be advanced through a tracheobronchial tree to the feeding bronchus B (upper airway) of the target area Cₛ, the lung compartment targeted for treatment or isolation. The catheter 10 comprises a shaft 12 having at least one lumen therethrough and an occlusion member 14 mounted near its distal end. The occlusion member 14 of the catheter 10 is adapted to seal the area between the catheter shaft 12 and the bronchial wall such that only a lumen inside the catheter which extends the entire length of the catheter is communicating with the airways distal to the seal. The seal, or isolation, may be accomplished by the use of the occlusion member 14, such as an inflatable member, attached to the distal tip of the catheter 10. Alternatively, a tip of the catheter can have an enlarged end or otherwise be adapted to seal in an airway without expansion or inflation.

On the opposite end of the catheter 10, external to the body of the patient, a one-way valve 16, a flow-measuring device 18 or/and a pressure sensor 20 are placed in series or otherwise as to communicate with the catheter's inside lumen. The one-way valve 16 prevents air from entering the target compartment Cₛ from atmosphere but allows free air movement from the target compartment Cₛ to atmosphere. When there is an absence of collateral channels connecting the targeted isolated compartment Cₛ to the rest of the lung, as illustrated in FIGS. 4A-4B, the isolated compartment Cₛ will unsuccessfully attempt to draw air from the catheter lumen during inspiration of normal respiration of the patient once the excess volume in the target compartment has been collapsed by the external pressure of the surrounding lung compartment. Hence, during exhalation no air is returned to the catheter lumen. In the presence of collateral channels, as illustrated in FIGS. 4C-4D, an additional amount of air is always available to the isolated compartment Cₛ during the inspiratory phase of each breath, namely the air traveling from the neighboring compartment(s) C through the collateral channels CH, which enables volumetric expansion of the isolated compartment Cₛ during inspiration, resulting during expiration in air movement away from the isolated compartment Cₛ to atmosphere through the catheter lumen and the collateral channels CH. Thus, air is expelled through the catheter lumen during each exhalation and will register as positive airflow on the flow-measuring device 18. This positive airflow through the catheter lumen provides an indication of whether or not there is collateral ventilation occurring in the targeted compartment Cₛ. It will be appreciated that in other embodiments, the one-way valve could be placed elsewhere on the catheter, including at or near the distal end.

The system of FIGS. 4A-4D can be used to determine the rate of oxygen uptake in a target lung compartment Cₛ in comparison to the rate in other portions of the lung or the entire remaining portion of the lung. The lung is isolated by inflation of the occlusion member 14 and allowed to deflate. After deflation has substantially stopped, as indicated for example by the flow detected by flow-measuring device 18 reaching zero (0), the rate of pressure decrease within the target lung compartment may be monitored over time. The rate at which the pressure decreases, as indicated by the pressure sensor 20, will be directly proportional to the oxygen uptake in the target compartment Cₛ and therefore be directly proportional to blood flow per unit of gas volume in the compartment.

The system of FIGS. 4A-4D can also be modified to help determine the directionality of flow through collateral channels between the target lung compartment and adjacent lung compartment(s). In particular, the catheter 10 can be modified so that flow through the lumen is blocked or the lumen is entirely absent. The occlusion member 14 will then fully occlude the target lung compartment Cₛ so that air neither enters nor leaves the compartment through the connecting upper airway. The target lung compartment is fully occluded with the catheter, and changes in pressure monitored over a plurality of respiratory cycles. If a pressure increase is measured by pressure sensor 20 is observed, it can be assumed that there is a net inflow of air from adjacent compartment(s) to the target lung compartment, indicating that there are collateral flow channels and that the collateral resistance in these flow channels is lower during inspiration than during expiration. Conversely, if a pressure decrease is observed, there are collateral flow channels having resistance which is greater during expiration than inspiration.

Determination of the existence and directionality of collateral channels between a target lung compartment and adjacent lung compartment(s) is information useful for both determining therapeutic treatment as well as determining the suitability of either diagnostic procedures performed according to the present invention. The existence of collateral channels which permit either entry or loss of air from the target lung compartment will also contraindicate other diagnostic procedures described herein which rely on maintaining a constant air volume within the lung compartment being diagnosed.

The system of FIGS. 4A-4D can also be used to determine the degree of hyperinflation and the compliance of a target lung compartment.

In a sixth aspect of the present invention, minimally invasive methods for evaluating the health of a target lung compartment relies on determining the blood flow per unit gas volume in the compartment. The isolation catheter 10 is used to isolate the target lung compartment Cₛ by deploying the occlusion member 14 as generally described above in connection with the other diagnostic protocols. A marker substance having a low blood solubility, such as sodium hexafluoride, is injected into systemic circulation, typically during apnea at mean lung volume. Although sodium hexafluoride is an example of a suitable marker, other low solubility gases may also be employed. Gas from the isolated lung compartment is sampled, typically through the lumen in the catheter 10, after a time sufficient for the blood concentration of the marker to reach equilibrium, typically after about 10 to 15 seconds. Concentration of the marker in other portions of the lung, typically in the rest of the lung as measured in exhaled air, is also determined. A concentration of the marker measured in the target lung compartment which is as great or greater than that displayed by other portions and/or in the entire remaining portion of the lungs is an indication that the blood flow per unit of gas volume is not compromised in the target lung compartment and that the target lung compartment is likely not a good candidate for therapeutic intervention. Conversely, if the measured blood flow per unit gas volume of the marker significantly less than that in other portions of the lung, the target lung volume appears to be a good candidate for therapy.

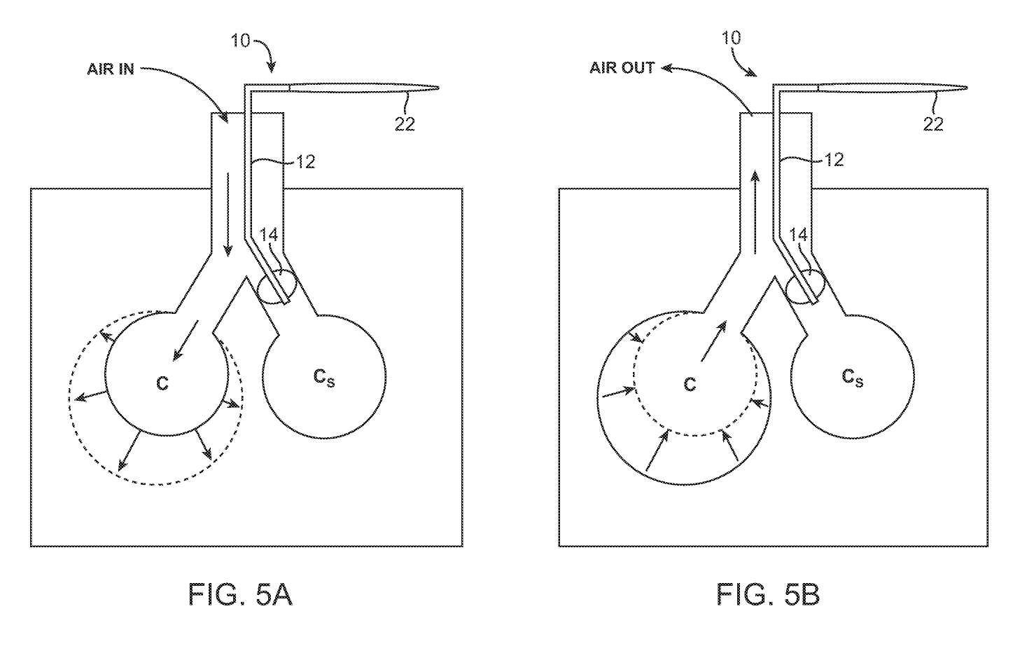

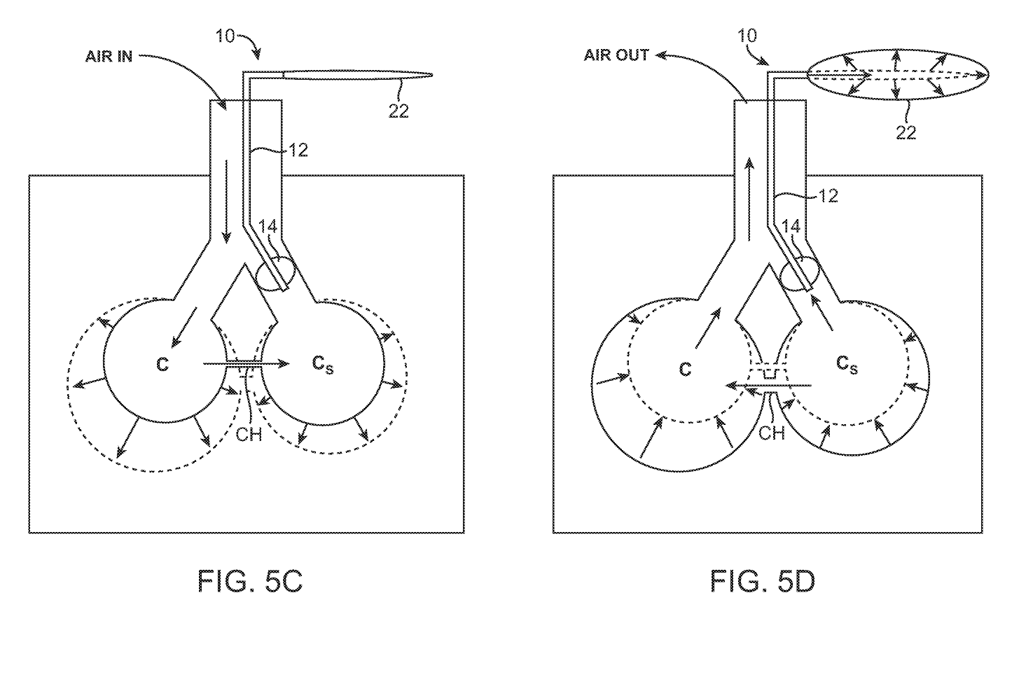

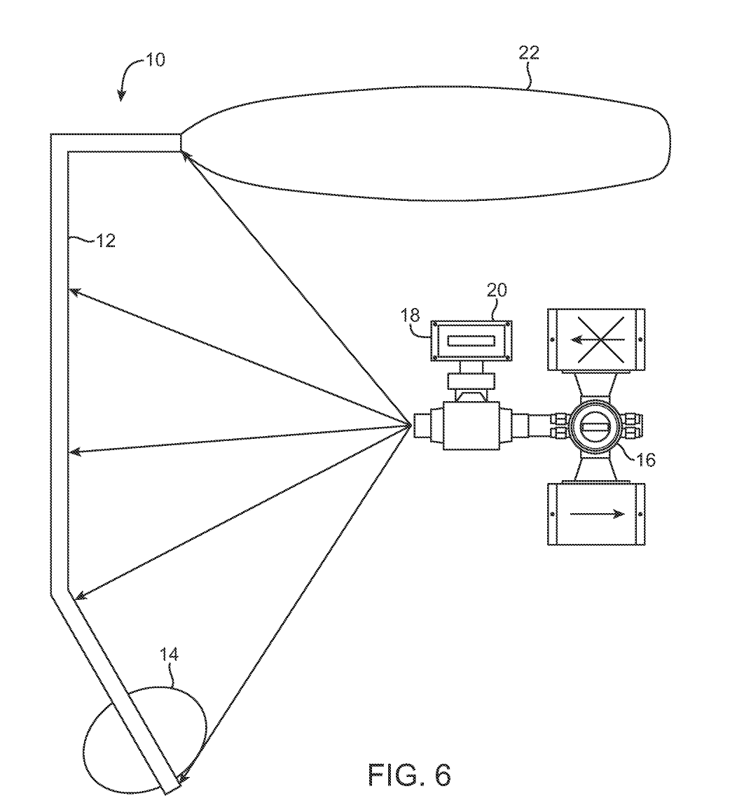

In other embodiments, the catheter 10 is connected with an accumulator or special container 22 as illustrated in FIGS. 5A-5D, 6. The container 22 has a very low resistance to airflow, such as but not limited to e.g. a very compliant bag or slack collection bag. The container 22 is connected to the external end or distal end 24 of the catheter 10 and its internal lumen extending therethrough in a manner in which the inside of the special container 22 is communicating only with the internal lumen. During respiration, when collateral channels are not present as illustrated in FIGS. 5A-5B, the special container 22 does not expand. The target compartment Cₛ is sealed by the isolation balloon 14 so that air enters and exits the non-target compartment C. During respiration, in the presence of collateral channels as illustrated in FIGS. 5C-5D, the special container 22 will initially increase in volume because during the first exhalation some portion of the airflow received by the sealed compartment Cₛ via the collateral channels CH will be exhaled through the catheter lumen into the external special container 22. The properties of the special container 22 are selected in order for the special container 22 to minimally influence the dynamics of the collateral channels CH, in particular a highly inelastic special container 22 so that it does not resist inflation. Under the assumption that the resistance to collateral ventilation is smaller during inspiration than during expiration, the volume in the special container 22 will continue to increase during each subsequent respiratory cycle because the volume of air traveling via collateral channels CH to the sealed compartment Cₛ will be greater during inspiration than during expiration, resulting in an additional volume of air being forced through the catheter lumen into the special container 22 during exhalation. This technique of measuring collateral flow in a lung compartment Cₛ is analogous to adding another lung compartment or lobe with infinitely large compliance, to the person's lungs, the added compartment being added externally.

Optionally, a flow-measuring device 18 or/and a pressure sensor 20 may be included, as illustrated in FIG. 6. The flow-measuring device 18 and/or the pressure sensor 20 may be disposed at any location along the catheter shaft 12 (as indicated by arrows) so as to communicate with the catheter's internal lumen. When used together, the flow-measuring device 18 and the pressure sensor 20 may be placed in series. A one-way valve 16 may also be placed in series with the flow-measuring device 18 or/and pressure sensor 20. It may be appreciated that the flow-measuring device 18 can be placed instead of the special container 22 or between the special container 22 and the isolated lung compartment, typically at but not limited to the catheter-special container junction, to measure the air flow rate in and out of the special container and hence by integration of the flow rate provide a measure of the volume of air flowing through the catheter lumen from/to the sealed compartment Cₛ.

It can be appreciated that measuring flow can take a variety of forms, such as but not limited to measuring flow directly with the flow-measuring device 18, and/or indirectly by measuring pressure with the pressure sensor 20, and can be measured anywhere along the catheter shaft 12 with or without a one-way valve 16 in conjunction with the flow sensor 18 and with or without an external special container 22.

In addition to determining the presence of collateral ventilation of a target lung compartment, the degree of collateral ventilation may be quantified by methods of the present invention. In one embodiment, the degree of collateral ventilation is quantified based on the resistance through the collateral system R_{coll}. R_{coll} can be determined based on the following equation:

$$\vert \frac{\overline{{P}_{b}}}{\overline{{Q}_{fm}}} \vert = {R}_{coll} + {R}_{saw} \qquad ( 1 )$$

where R_{coll} constitutes the resistance of the collateral channels, R_{saw} characterizes the resistance of the small airways, and P̅_̅{̅b̅}̅ and Q̅_̅{̅f̅m̅}̅ represent the mean pressure and the mean flow measured by a catheter isolating a target lung compartment in a manner similar to the depictions of FIGS. 4A-4D.

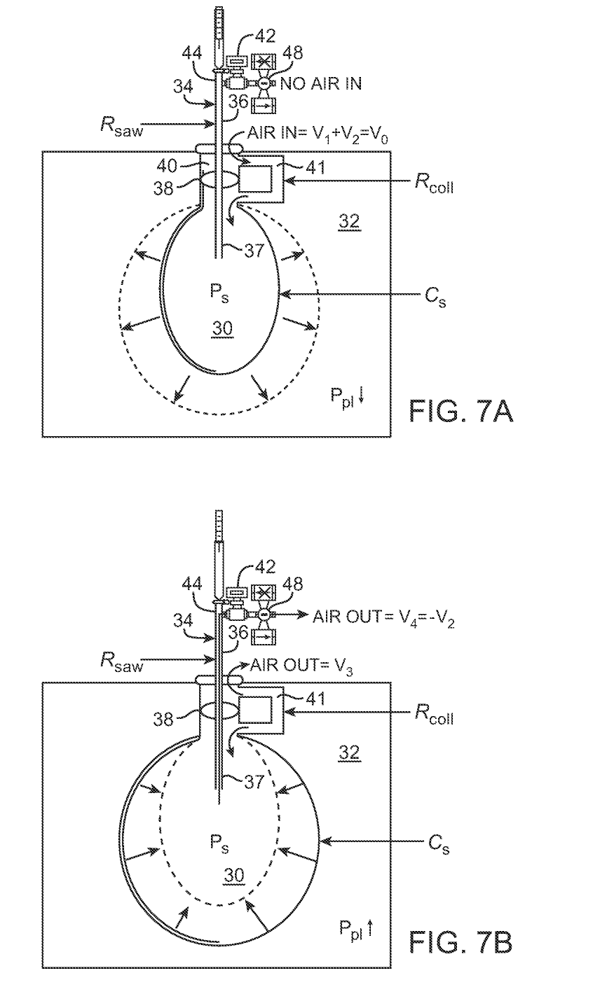

For the sake of simplicity, and as a means to carry out a proof of principle, FIGS. 7A-7B depict a graphical representation of a simplified collateral system of a target lung compartment Cₛ. A single elastic compartment 30 represents the target lung compartment Cₛ and is securely positioned inside a chamber 32 to prevent any passage of air between the compartment 30 and the chamber 32. The chamber 32 can be pressurized to a varying negative pressure relative to atmosphere, representing the intrathoracic pressure Pₚₗ. The elastic compartment 30, which represents the target compartment in the lung Cₛ, communicates with the atmospheric environment through passageway 40. In addition, the elastic compartment 30 also communicates with the atmospheric environment through collateral pathway 41, representing collateral channels CH of the target compartment of the lung Cₛ.

A catheter 34 is advanceable through the passageway 40, as illustrated in FIGS. 7A-7B. The catheter 34 comprises a shaft 36, an inner lumen 37 therethrough and an occlusion member 38 mounted near it's distal end. The catheter 34 is specially equipped to seal the area between the catheter shaft 36 and the passageway 40 such that only the lumen 37 inside the catheter 34, which extends the length of the catheter 34, allows for direct communication between the compartment 30 and atmosphere. On the opposite end of the catheter 34, a flow-measuring device 42 and a pressure sensor 44 are placed in series to detect pressure and flow in the catheter's inside lumen 37. A one-way valve 48 positioned next to the flow measuring device 42 allows for the passage of air in only one direction, namely from the compartment 30 to atmosphere. The flow measuring device 42, the pressure sensor device 44 and the one-way valve 48 can be placed anywhere along the length of the catheter lumen, typically at but not limited to the proximal end of the catheter shaft 36. It should be appreciated that measuring pressure inside the compartment 30 can be accomplished in a variety of forms, such as but not limited to connecting the pressure sensor 44 to the catheter's inside lumen 37. For instance, it can also be accomplished by connecting the pressure sensor 44 to a separate lumen inside the catheter 34, which extends the entire length of the catheter 34 communication with the airways distal to the seal.

At any given time, the compartment 30 may only communicate to atmosphere either via the catheter's inside lumen 37 representing R_{saw} and/or the collateral pathway 41 representing R_{coll}. Accordingly, during inspiration, as illustrated in FIG. 7A, Pₚₗ becomes increasingly negative and air must enter the compartment 30 solely via collateral channels 41. Whereas during expiration, illustrated in FIG. 7B, air may leave via collateral channels 41 and via the catheter's inside lumen 37.

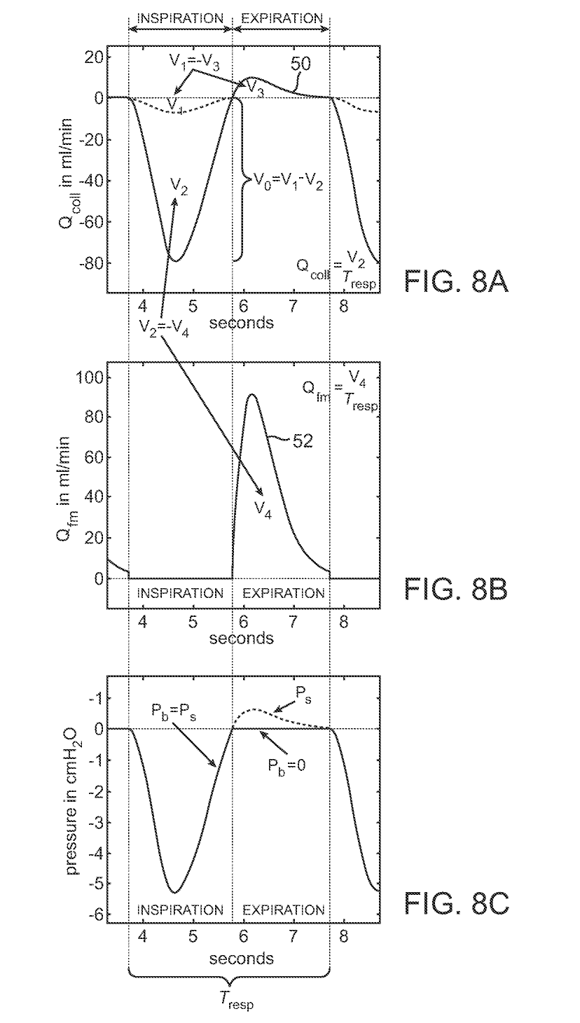

FIGS. 8A-8C illustrate measurements taken from the system of FIGS. 7A-7B during inspiration and expiration phases. FIG. 8A illustrates a collateral flow curve 50 reflecting the flow Q_{coll} through the collateral pathway 41. FIG. 8B illustrates a catheter flow curve 52 reflecting the flow Q_{fm} through the flow-measuring device 42. During inspiration, air flows through the collateral pathway 41 only; no air flows through the flow-measuring device 42 since the one-way valve 48 prevents such flow. Thus, FIG. 8A illustrates a negative collateral flow curve 50 and FIG. 8B illustrates a flat, zero-valued catheter flow curve 52. During expiration, a smaller amount of air, as compared to the amount of air entering the target compartment Cs during inspiration, flows back to atmosphere through the collateral pathway 41, as illustrated by the positive collateral flow curve 50 of FIG. 8A, while the remaining amount of air flows through the catheter lumen 37 back to atmosphere, as illustrated by the positive catheter flow curve 52 of FIG. 8B.

The volume of air flowing during inspiration and expiration can be quantified by the areas under the flow curves 50, 52. The total volume of air V₀ entering the target compartment 30 via collateral channels 41 during inspiration can be represented by the colored area under the collateral flow curve 50 of FIG. 8A. The total volume of air V₀ may be denoted as V₀=V₁+V₂, whereby V₁ is equal to the volume of air expelled via the collateral channels 41 during expiration (indicated by the grey-colored area under the collateral flow curve 50 labeled V₃), and V₂ is equal to the volume of air expelled via the catheter's inside lumen 37 during expiration (indicated by the colored area under the catheter flow curve 52 of FIG. 8B labeled V₄).

The following rigorous mathematical derivation demonstrates the validity of theses statements and the relation stated in Eq. 1:

Conservation of mass states that in the short-term steady state, the volume of air entering the target compartment 30 during inspiration must equal the volume of air leaving the same target compartment 30 during expiration, hence
V₀=−(V₃+V₄) (2)
Furthermore, the mean rate of air entering and leaving the target compartment solely via collateral channels during a complete respiratory cycle (Tᵣₑₛₚ) can be determined as

$$\overline{{Q}_{coll}} = \frac{{V}_{0} + {V}_{3}}{{T}_{resp}} = \frac{{V}_{2}}{{T}_{resp}} \qquad ( 3 )$$

where V₂ over Tᵣₑₛₚ represents the net flow rate of air entering the target compartment 30 via the collateral channels 41 and returning to atmosphere through a different pathway during Tᵣₑₛₚ. Accordingly, V₂ accounts for a fraction of V₀, the total volume of air entering the target compartment 30 via collateral channels 41 during Tᵣₑₛₚ, hence V₀ can be equally defined in terms of V₁ and V₂ as
V₀=V₁+V₂ (4)
where V₁ represents the amount of air entering the target compartment 30 via the collateral channels 41 and returning to atmosphere through the same pathway. Consequently, substitution of V₀ from Eq. 4 into Eq. 3 yields
V₁=−V₃ (5)
and substitution of V₀ from Eq. 2 into the left side of Eq. 4 following substitution of V₁ from Eq. 5 into the right side of Eq. 4 results in
−V₄=V₂ (6)
Furthermore, the mean flow rate of air measured at the flowmeter 42 during Tᵣₑₛₚ can be represented as

$$\overline{{Q}_{fm}} = \frac{{V}_{4}}{{T}_{resp}} \qquad ( 7 )$$

where substitution of V₄ from Eq. 6 into Eq. 7 yields

$$\overline{{Q}_{fm}} = - \frac{{V}_{2}}{{T}_{resp}} = - \overline{{Q}_{coll}} \qquad ( 8 )$$

Ohms's law states that in the steady state
P̅ₛ̅= Q̅_̅{̅c̅o̅l̅l̅}̅·R_{coll} (9)
where P̅₅̅ represents the mean inflation pressure in the target compartment required to sustain the continuous passage of Q̅_̅{̅c̅o̅l̅l̅}̅ through the resistive collateral channels represented by R_{coll}. Visual inspection of the flow and pressure signals (FIG. 8C) within a single Tᵣₑₛₚ shows that during the inspiratory time, P_{b} corresponds to Pₛ since no air can enter or leave the isolated compartment 30 via the catheter's inside lumen 37 during the inspiratory phase. During expiration, however, P_{b}=0 since it is measured at the valve opening where pressure is atmospheric, while Pₛ must still overcome the resistive pressure losses produced by the passage of Q_{fm} through the long catheter's inside lumen 37 represented by R_{saw} during the expiratory phase effectively making P̅ₛ̅ less negative than P̅_̅{̅b̅}̅ by Q̅_̅{̅f̅m̅}̅ ·R_{saw}. Accordingly
P̅ₛ̅= P̅_̅{̅b̅}̅+ Q̅_̅{̅f̅m̅}̅·R_{saw} (10)
and substitution of Pₛ from Eq. 9 into Eq. 10 results in
P̅_̅{̅b̅}̅= Q̅_̅{̅c̅o̅l̅l̅}̅·R_{coll}− Q̅_̅{̅f̅m̅}̅·R_{saw} (11)
after subsequently solving for P̅_̅{̅b̅}̅. Furthermore, substitution of Q̅_̅{̅c̅o̅l̅l̅}̅ from Eq. 8 into Eq. 11 yields
P̅_̅{̅b̅}̅=− Q̅_̅{̅f̅m̅}̅·(R_{coll}+R_{saw}) (12)
and division of Eq. 12 by Q̅_̅{̅f̅m̅}̅ finally results in

$$\frac{\overline{{P}_{b}}}{\overline{{Q}_{fm}}} = - ( {R}_{coll} + {R}_{saw} ) \qquad ( 13 )$$

where the absolute value of Eq. 13 leads back to the aforementioned relation originally stated in Eq. 1.

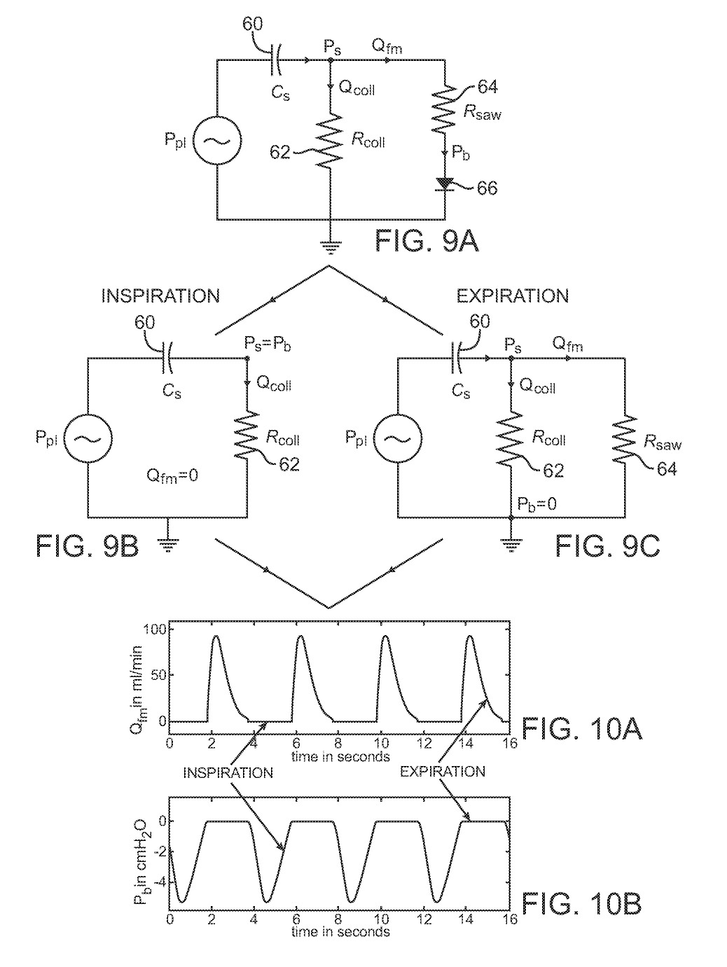

The system illustrated in FIGS. 7A-7B can be represented by a simple circuit model as illustrated in FIGS. 9A-9C. The air storage capacity of the alveoli confined to the isolated compartment 30 representing Cₛ is designated as a capacitance element 60. The pressure gradient (Pₛ-P_{b}) from the alveoli to atmosphere via the catheter's inside lumen 37 is caused by the small airways resistance, R_{saw}, and is represented by resistor 64. The pressure gradient from the alveoli to atmosphere through the collateral channels is generated by the resistance to collateral flow, R_{coll}, and is represented by resistor 62.

Accordingly, the elasticity of the isolated compartment 30 is responsible for the volume of air obtainable solely across R_{coll} during the inspiratory effort and subsequently delivered back to atmosphere through R_{saw}, and R_{coll} during expiration. Pressure changes during respiration are induced by the variable pressure source, Pₚₗ representing the varying negative pleural pressure within the thoracic cavity during the respiratory cycle. An ideal diode 66 represents the one-way valve 48, which closes during inspiration and opens during expiration. Consequently, as shown in FIGS. 10A-10B, the flow measured by the flow meter (Q_{fm}) is positive during expiration and zero during inspiration, whereas the pressure recorded on the pressure sensor (P_{b}) is negative during inspiration and zero during expiration.

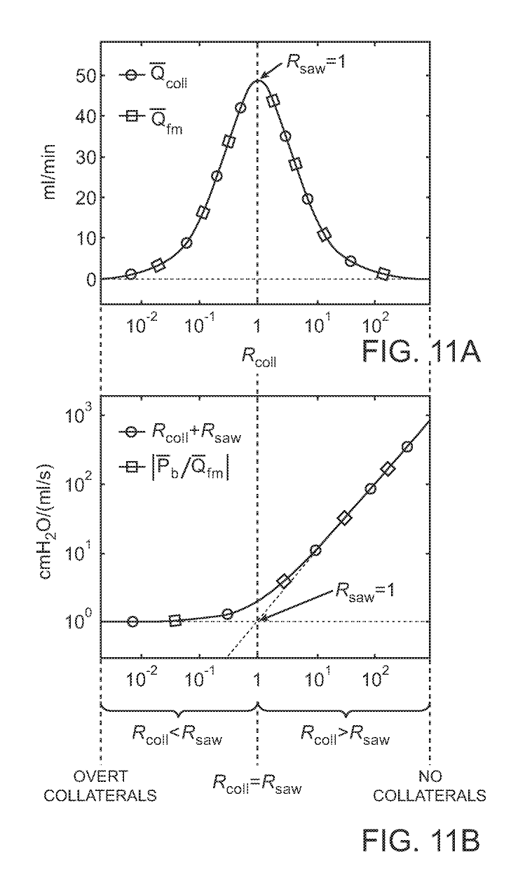

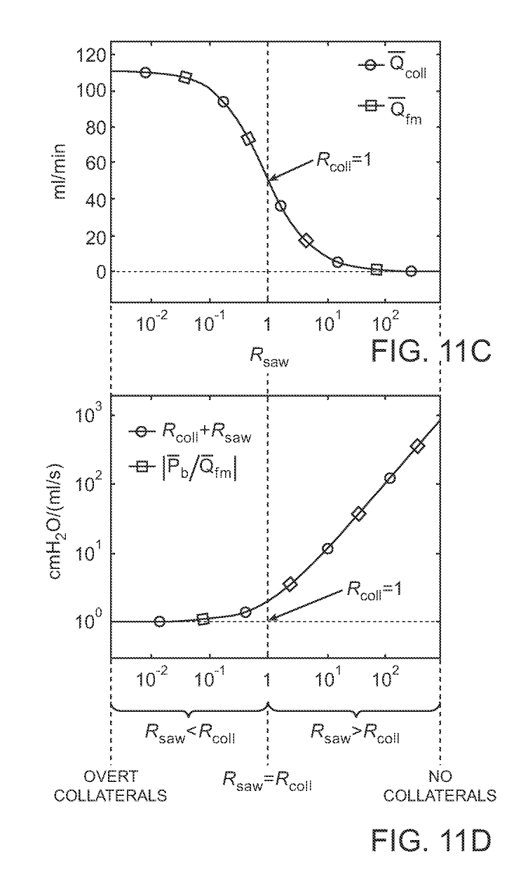

Evaluation of Eqs. 1 & 8 by implementation of a computational model of the collateral system illustrated in FIGS. 7A-7B and FIGS. 9A-9C yields the graphical comparisons presented in FIGS. 11A-11D. FIG. 11A displays the absolute values of mean Q_{fm} (| Q̅_̅{̅f̅m̅}̅|) and mean Q_{coll} (| Q̅_̅{̅c̅o̅l̅l̅}̅|) while the FIG. 11B shows the model parameters R_{coll}+R_{saw} plotted together with | P̅_̅{̅b̅}̅/ Q̅_̅{̅c̅o̅l̅l̅}̅| as a function of R_{coll}. The values denote independent realizations of computer-generated data produced with different values of R_{coll} while R_{saw} is kept constant at 1 cmH₂O/(ml/s). FIG. 11A displays the absolute values of | Q̅_̅{̅f̅m̅}̅| and | Q̅_̅{̅c̅o̅l̅l̅}̅| while FIG. 11C shows the model parameters R_{coll}+R_{saw} plotted together with | P̅_̅{̅b̅}̅/ Q̅_̅{̅c̅o̅l̅l̅}̅| as a function of R_{saw}. The values denote independent realizations of computer-generated data produced with different values of R_{saw} while R_{coll} is kept constant at 1 cmH₂O/(ml/s). It becomes quite apparent from FIGS. 11A-11B that the flow is maximal when R_{coll}≈R_{saw} and diminishes to zero as R_{coll} approaches the limits of either “overt collaterals” or “no collaterals”. Accordingly, small measured flow Q_{fm} can mean both, very small and very large collateral channels and hence no clear-cut decision can be made regarding the existence of collateral ventilation unless R_{coll}+R_{saw} is determined as | P̅_̅{̅b̅}̅/ Q̅_̅{̅f̅m̅}̅|. The reason for this is that when R_{coll} is very small compared to R_{saw}, all gas volume entering the target compartment via the collateral channels leaves via the same pathway and very little gas volume is left to travel to atmosphere via the small airways as the isolated compartment empties. The measured pressure P_{b}, however, changes accordingly and effectively normalizes the flow measurement resulting in an accurate representation of R_{coll}+R_{saw}, which is uniquely associated with the size of the collateral channels and the correct degree of collateral ventilation.

Similarly, FIGS. 11C-11D supplement FIGS. 11A-11B as it shows how the measured flow Q_{fm} continuously diminishes to zero as R_{saw} becomes increasingly greater than R_{coll} and furthermore increases to a maximum, as R_{saw} turns negligible when compared to R_{coll}. When R_{saw} is very small compared to R_{coll}, practically all gas volume entering the target compartment via the collateral channels travels back to atmosphere through the small airways and very little gas volume is left to return to atmosphere via the collateral channels as the isolated compartment empties. Thus, determination of | P̅_̅{̅b̅}̅/ Q̅_̅{̅f̅m̅}̅| results in an accurate representation of R_{coll}+R_{saw} regardless of the underlying relation amongst R_{coll} and R_{saw}. In a healthy human, resistance through collateral communications, hence R_{coll}, supplying a sublobar portion of the lung is many times (10-100 times) as great as the resistance through the airways supplying that portion, R_{saw} (Inners 1979, Smith 1979, Hantos 1997, Suki 2000). Thus in the normal individual, R_{coll} far exceeds R_{saw} and little tendency for collateral flow is expected. In disease, however, this may not be the case (Hogg 1969, Terry 1978). In emphysema, R_{saw} could exceed R_{coll} causing air to flow preferentially through collateral pathways.

Therefore, the above described models and mathematical relationships can be used to provide a method which indicates the degree of collateral ventilation of the target lung compartment of a patient, such as generating an assessment of low, medium or high degree of collateral ventilation or a determination of collateral ventilation above or below a clinical threshold. In some embodiments, the method also quantifies the degree of collateral ventilation, such generating a value which represents R_{coll}. Such a resistance value indicates the geometric size of the collateral channels in total for the lung compartment. Based on Poiseuille's Law with the assumption of laminar flow,
R∝(η×L)/r⁴ (14)
wherein η represents the viscosity of air, L represents the length of the collateral channels and r represents the radius of the collateral channels. The fourth power dependence upon radius allows an indication of the geometric space subject to collateral ventilation regardless of the length of the collateral channels.

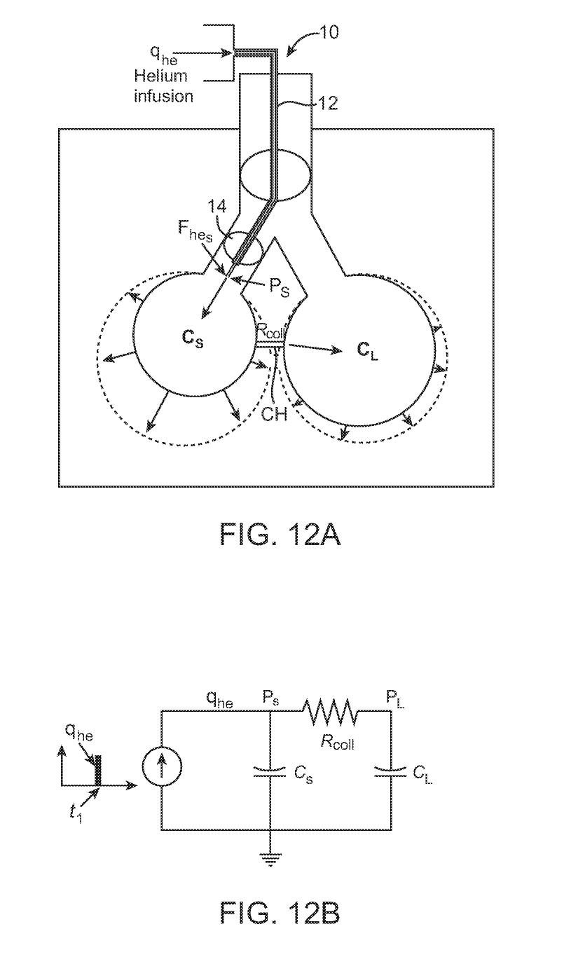

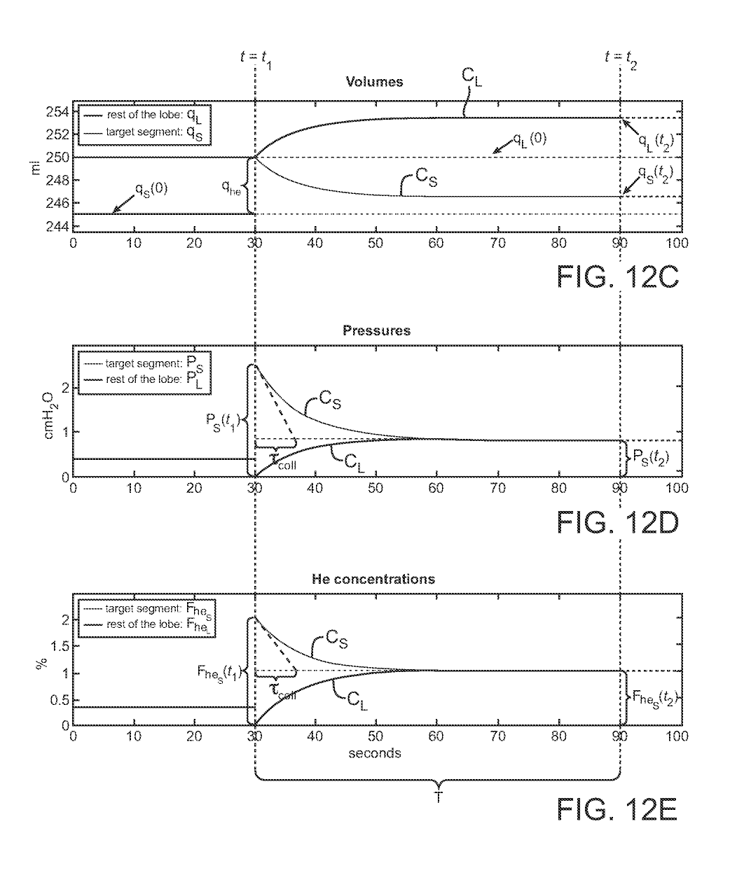

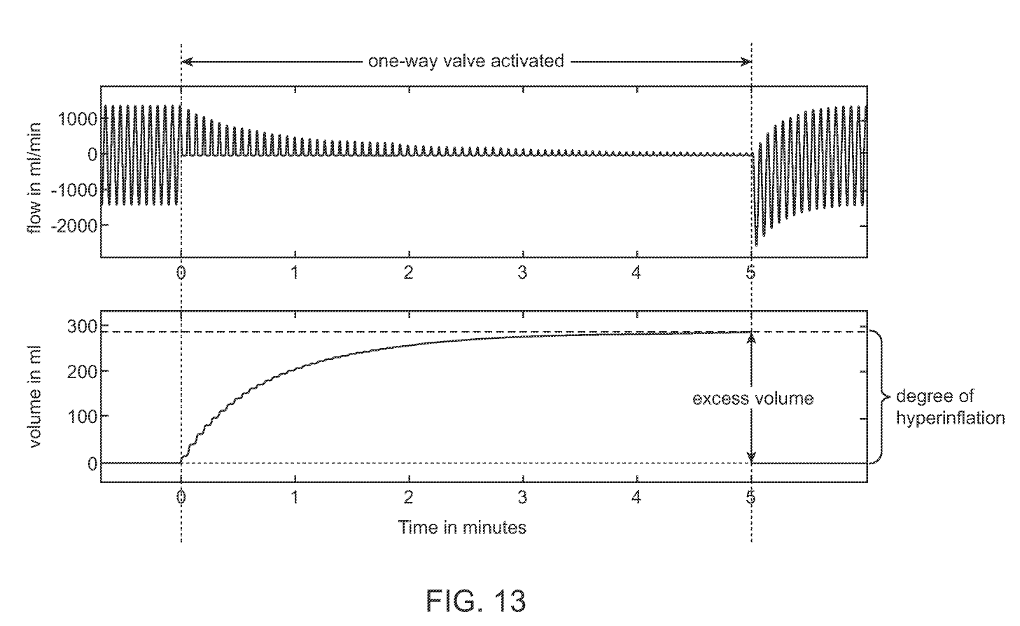

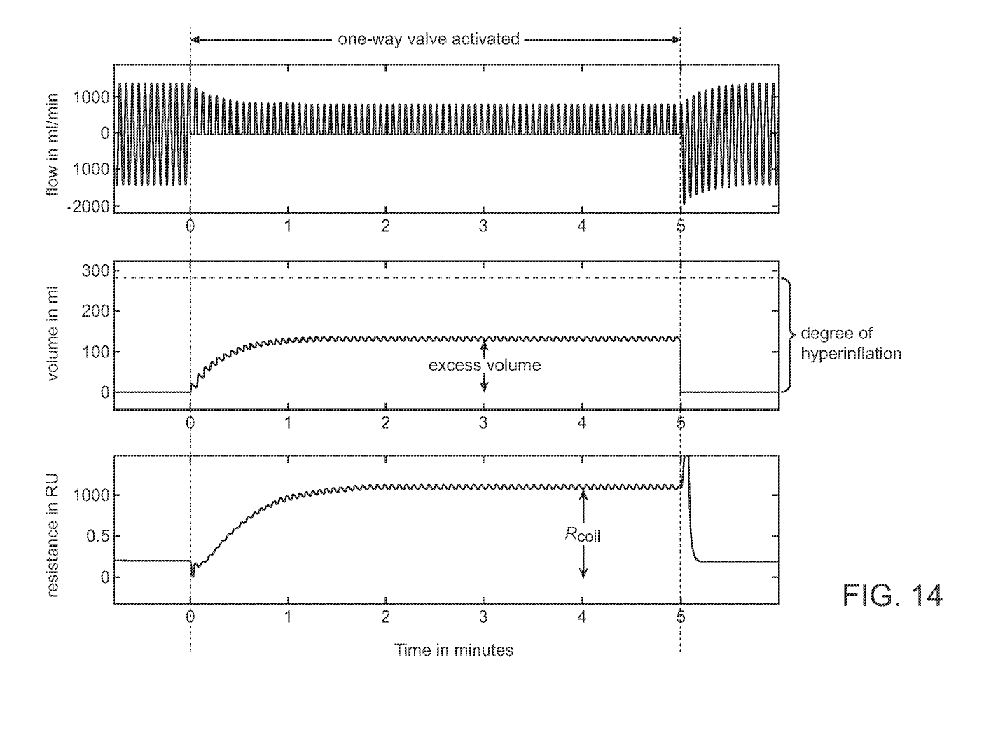

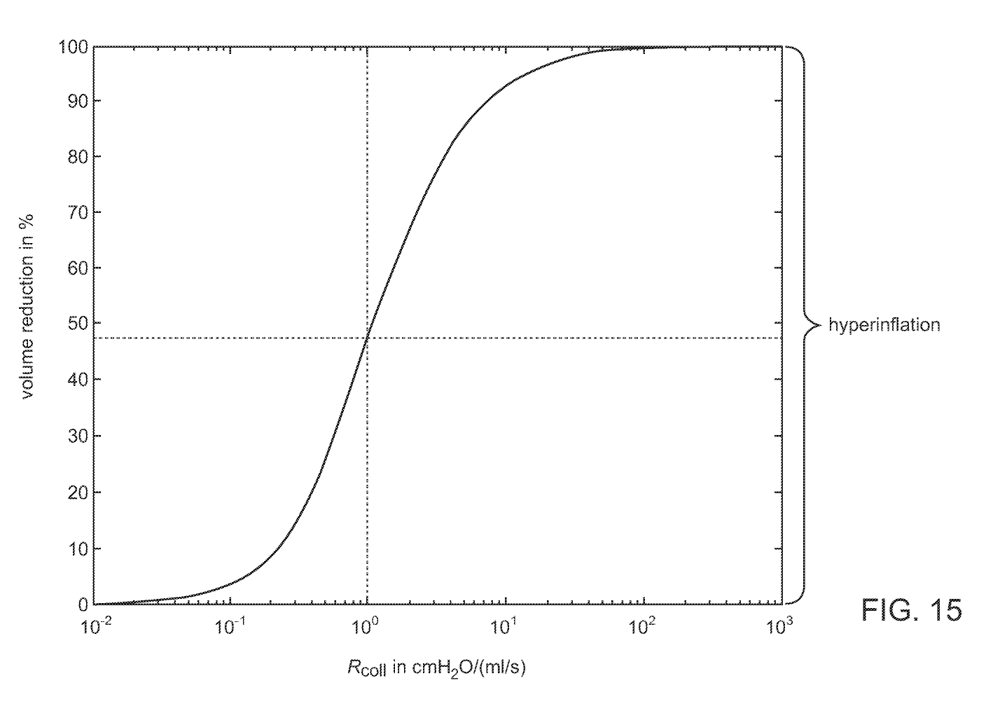

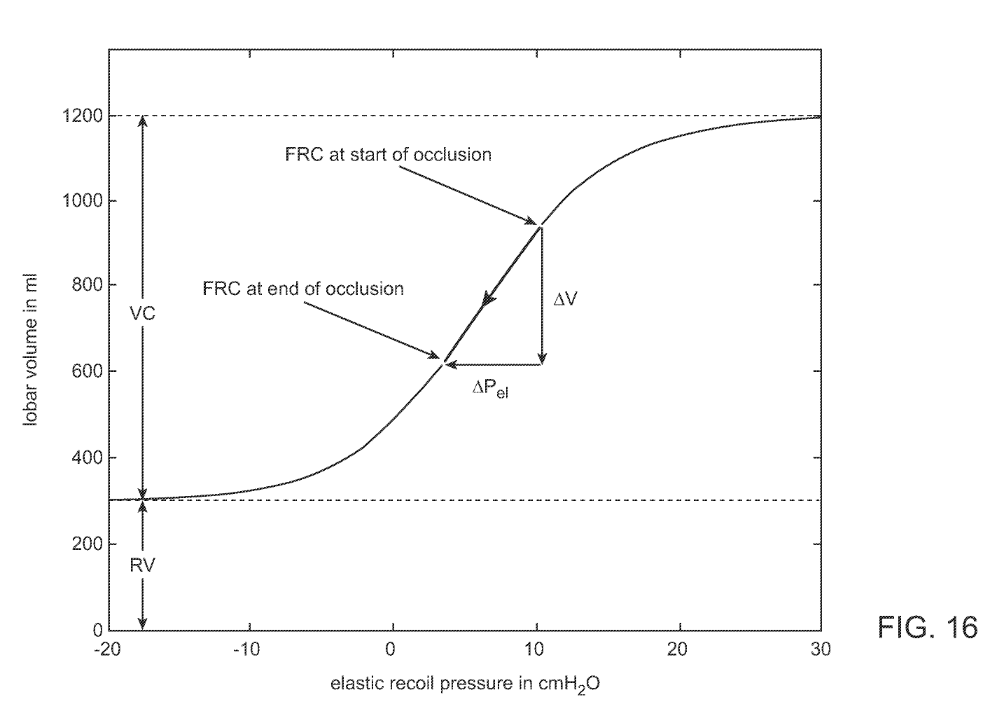

FIG. 12A illustrates a two-compartment model which is used to generate a method quantifying the degree of collateral ventilation, including a) determining the resistance to segmental collateral flow R_{coll}, b) determining the state of segmental compliance Cₛ, and c) determining the degree of segmental hyperinflation qₛ. Again, Cₛ characterizes the compliance of the target compartment or segment. C_{L} represents the compliance of the rest of the lobe. R_{coll} describes the resistance to the collateral airflow. FIG. 12B provides an electrical circuit analog model. In this example, at time t=t₁, approximately 5-10ml of 100% inert gas such as He (qₕₑ) is infused. After a period of time, such as one minute, the pressure (Pₛ) & the fraction of He (Fₕₑ_{ₛ}) are measured.

The dynamic behavior of the system depicted in FIGS. 12A-12B can be described by the time constant τ_{coll}

$${τ}_{coll} = {R}_{coll} \cdot \frac{{C}_{S} {C}_{L}}{\underbrace{{C}_{S} + {C}_{L}}_{{c}_{a}}} \qquad ( 15 )$$

At time t₁=30 s, a known fixed amount of inert gas (qₕₑ: 5-10 ml of 100% He) is rapidly injected into the target compartment Cₛ, while the rest of the lobe remains occluded, and the pressure (Pₛ) and the fraction of He (Fₕₑ_{_{S}}) are measured in the target segment for approximately one minute (T=60 s). FIGS. 12C-12E illustrate the resulting time changes in volumes, pressures and gas concentrations in the target compartment Cₛ and the rest of the lobe C_{L}. Eqs. 16-21 state the mathematical representation of the lung volumes, pressures and gas concentrations at two discrete points in time, t₁ and t₂.

$$\begin{matrix}{q}_{s} ( {t}_{1} ) = {q}_{s} ( 0 ) + {q}_{he} & ( 16 ) \\ {q}_{s} ( {t}_{2} ) + {q}_{L} ( {t}_{2} ) = {q}_{s} ( 0 ) + {q}_{L} ( 0 ) + {q}_{he} & ( 17 ) \\ {P}_{s} ( {t}_{1} ) = \frac{{q}_{he}}{{C}_{s}} & ( 18 ) \\ {P}_{s} ( {t}_{2} ) = \frac{{q}_{he}}{( {C}_{s} + {C}_{L} )} & ( 19 ) \\ {F}_{{he}_{s}} ( {t}_{1} ) = \frac{{q}_{he}}{{q}_{s} ( {t}_{1} )} & ( 20 ) \\ {F}_{{he}_{s}} ( {t}_{2} ) = \frac{{q}_{he}}{{q}_{s} ( {t}_{1} ) + {q}_{L} ( {t}_{2} )} & ( 21 )\end{matrix}$$

As a result, the following methods may be performed for each compartment or segment independently: 1) Assess the degree of segmental hyperinflation, 2) Determine the state of segmental compliance, 3) Evaluate the extent of segmental collateral communications.

Segmental Hyperinflation

The degree of hyperinflation in the target segment, qₛ(0), can be determined by solving Eq. 16 for qₛ(0) and subsequently substituting qₛ(t₁) from Eq. 20 into Eq. 16 after appropriate solution of Eq. 20 for qₛ(t₁) as

$${q}_{S} ( 0 ) = {q}_{he} \cdot ( \frac{1 - {F}_{{he}_{s}} ( {t}_{1} )}{{F}_{{he}_{s}} ( {t}_{1} )} ) \qquad ( 22 )$$

Segmental Compliance

The state of compliance in the target segment, C_{S}, can be determined simply by solving Eq. 18 for C_{S} as

$${C}_{S} = \frac{{q}_{he}}{{P}_{S} ( {t}_{1} )} \qquad ( 23 )$$

Segmental Collateral Resistance

A direct method for the quantitative determination of collateral system resistance in lungs, has been described above. Whereas, the calculation below offers an indirect way of determining segmental collateral resistance.

The compliance of the rest of the lobe, C_{L}, can be determined by solving Eq. 19 for C_{L} and subsequently substituting C_{S} with Eq. 23. Accordingly

$${C}_{L} = {q}_{he} \cdot \frac{{P}_{S} ( {t}_{1} ) - {P}_{S} ( {t}_{2} )}{{P}_{S} ( {t}_{1} ) {P}_{S} ( {t}_{2} )} \qquad ( 24 )$$

As a result, the resistance to collateral flow/ventilation can alternatively be found by solving Eq. 15 for R_{coll} and subsequent substitution into Eq. 15 of C_{S} from Eq. 24 and C_{L} from Eq. 25 as

$${R}_{coll} = \frac{{τ}_{coll}}{{C}_{eff}} \qquad ( 25 )$$

where C_{eff} is the effective compliance as defined in Eq. 15.
Additional Useful Calculation for Check and Balances of All Volumes

The degree of hyperinflation in the rest of the lobe, hence q_{L}(0), can be determined by solving Eq. 17 for q_{L}(0) and subsequently substituting qₛ(t₂)+q_{L}(t₂) from Eq. 21 into Eq. 17 after appropriate solution of Eq. 21 for q_{S}(t₂)+q_{L}(t₂). Thus

$${q}_{L} ( 0 ) = {q}_{he} \cdot ( \frac{{F}_{{he}_{S}} ( {t}_{1} ) - {F}_{{he}_{S}} ( {t}_{2} )}{{F}_{{he}_{S}} ( {t}_{1} ) {F}_{{he}_{S}} ( {t}_{2} )} ) \qquad ( 26 )$$

Equation 26 provides an additional measurement for check and balances of all volumes at the end of the clinical procedure.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A method for determining the extent of hyperinflation of a lung compartment, said method comprising:
occluding the lung compartment with a catheter so that all air expelled from the compartment passes out through the catheter; and
measuring the total amount of air expelled from the compartment from the time of initial occlusion until flow from the compartment substantially stops.

2. A method as in claim 1, wherein occluding comprises expanding an occlusion structure on the catheter at an airway leading to the lung compartment.

3. A method as in claim 1, wherein measuring the total amount of air comprises collecting the air in a bag.

4. A method as in claim 1, further comprising measuring the rate of air flow from the compartment to determine when the air flow substantially stops.

5. A method for determining gas exchange between an isolated lung compartment and blood, said method comprising:
occluding the lung compartment with a catheter which allows air to be expelled from the compartment but not to enter the compartment;
after air flow from the compartment through the catheter ceases, measuring gas pressure within the compartment, wherein a change in gas pressure is a measure of gas exchange in the lung compartment.

6. A method as in claim 5, wherein occluding comprises expanding an occlusion structure on the catheter at an airway leading to the lung compartment.

7. A method as in claim 6, wherein the catheter comprises a one-way valve which allows air to be expelled from the compartment but not to enter the compartment.

8. A method as in claim 5, wherein gas pressure is measured with a transducer on the catheter.

9. A method for determining directionality of collateral channels communicating with a lung compartment, said method comprising:
isolating the lung compartment so that there is no flow in or out through the connecting airway; and
measuring pressure within the isolated lung compartment over a plurality of respiratory cycles;
wherein an increase in pressure indicates that the collateral channels have a higher resistance to outflow than inflow and wherein a decrease in pressure indicates that the collateral channels have a lower resistance to outflow than to inflow.

10. A method as in claim 9, wherein isolating the lung compartment comprises expanding an occlusion structure on a catheter at an airway leading to the lung compartment.

11. A method as in claim 9, wherein pressure is measured with a transducer on the catheter.

12. A method for assessing blood flow in a lung compartment, said method comprising:
isolating the lung compartment;
injecting into systemic circulation a marker with low blood solubility that will be released into the lung;
measuring a first concentration of the marker in the lung compartment t and a second concentration of the marker in another part of the lung after systemic concentration of the marker has reached equilibrium; and
comprising the marker concentration in the compartment with the marker concentration in the other part of the lung, where a lower gas concentration indicates less blood perfusion.

13. A method as in claim 12, wherein the marker is injected during apnea at mean lung volume.

14. A method as in claim 12, wherein the marker is sulfur hexafluoride.

15. A method as in claim 12, wherein the second concentration is measured in gas exhaled from the rest of the lung.

16. A method determining the compliance of a lung compartment, said method comprising:
measuring a characteristic pressure-volume curve of an isolated lung compartment; and
determining compliance based on the slope of the measured characteristic pressure-volume curve.

17. A method as in claim 16, wherein measuring a characteristic pressure-volume curve comprises determining the difference between a pressure change in the isolated lung compartment and a change in pleural pressure, and measuring the corresponding volume change in the isolated lung compartment.

18. A method as in claim 17, wherein the pressure change in the isolated lung compartment is measured by or through a catheter open to the lung compartment.

19. A method as in claim 18, wherein the change in pleural pressure is measured by an esophageal balloon catheter.

20. A method for determining gas exchange.

21. A method as in claim 5, wherein a decrease in gas pressure is detected as a measure of oxygen uptake by the blood.

22. A method as in claim 5, wherein an increase in gas pressure is detected as a measure of carbon dioxide release from the blood.
